(19) United States
(12) Patent Application Publication
(10) Pub. No.: US 2003/0153608 A1
(43) Pub. Date: Aug. 14, 2003

(54) Torasemide-containing pharmaceutical preparations

(76) Inventors: Markus Maegerlein, Mannheim (DE); Thomas Hantke, Mannheim (DE); Jorg Breitenbach, Mannheim (DE); Jorg Rosenberg, Ellerstatd (DE)

Correspondence Address: Keil & Weinkauf, 1350 Connecticut Avenue NW, Washington, DC, 20036, US

(21) Appl. No.: 10/221,205

(22) PCT Filed: Mar. 15, 2001
(86) PCT No.: PCT/EP01/02940

(30) Foreign Application Priority Data
Mar. 17, 2000 (DE) 10013289.8

(51) Int. Cl. A61K 31/44

(52) U.S. Cl. 514/347

(57) ABSTRACT

Stable pharmaceutical preparations, comprising torasemide in noncrystalline form.

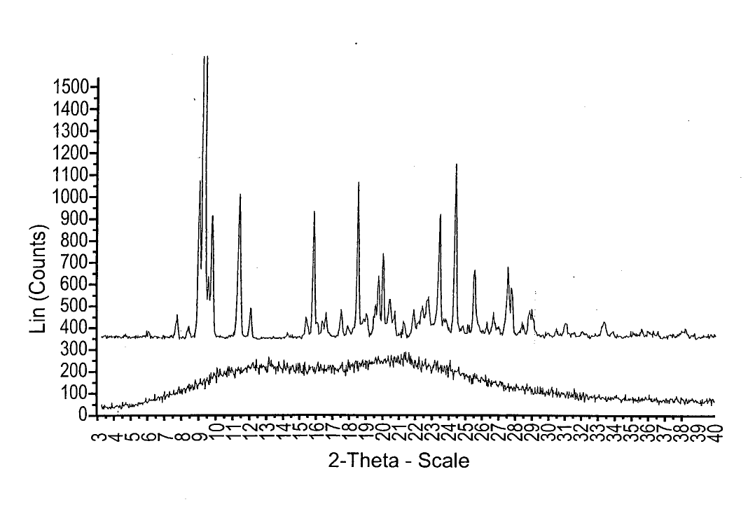

The present invention relates to preparations of the active compound torasemide, in which the torasemide is present in essentially noncrystalline form. The invention furthermore relates to a process for the production of such preparations, and pharmaceutical forms comprising such preparations.

Torasemide (1-isopropyl-3-[(4-m-toluidino-3-pyridyl)sulfonyl]urea is a loop diuretic, which is employed in different doses for the treatment of hypertension, edema and renal insufficiency.

Three polymorphic forms of torasemide are known to date. The modifications I and II are described in Acta Cryst., 1978, pp. 2659-2662 and Acta Cryst., 1987, pp. 1304-1310. US Re 34,672 and 34,680 disclose that the modification II rapidly rearranges to the modification I.

U.S. Pat. No. 5,914,336 discloses a further modification III which can also be employed as a mixture with the modification I.

WO 93/00097 describes storage-stable formulations of torasemide in which the active compound is preferably employed in the form of the modification I.

This process conceals a number of disadvantages. According to the synthesis, the active compound must be reprocessed in a complicated and expensive manner in order to obtain the required modification I. Furthermore, the active compound must be present in a closely defined particle size (90%≦96 μm and 50%≦48 μm) in order that the desired rapid release of active compound is achieved. This makes appropriate grinding and classification steps necessary.

It is an object of the present invention to make available pharmaceutical preparations comprising torasemide which help to avoid the disadvantages accompanying the occurrence of polymorphic forms.

Accordingly, we have found that this object is achieved by storage-stable solid or semisolid preparations in which the torasemide is present in essentially noncrystalline form. Preferably, the preparations are a so-called “solid solution”. The preparations, however, can also contain amorphous agglomerates dispersed homogeneously in the binder matrix, the size of such agglomerates preferably being in the region of ≦1 μm.

Essentially noncrystalline within the meaning of this invention means that not more than 5%, preferably not more than 2%, of the active compound is present in the form of crystals. Particularly preferred preparations are those which are free of crystalline active compound.

The concept of the solid solution is known to the person skilled in the art and essentially describes molecularly disperse systems in which the active compound is homogeneously dispersed in a binder matrix serving as a solvent.

Within the meaning of this invention, torasemide also means the corresponding pharmacologically acceptable salts such as, for example, salts with organic acids such as acetic acid, maleic acid, fumaric acid, malic acid, tartaric acid, ascorbic acid, methanesulfonic acid, citric acid or with inorganic acids such as hydrochloric acid, hydrobromic acid, phosphoric acid or sulfuric acid.

According to the invention, the binder matrix is at least partially soluble or swellable in aqueous systems.

Suitable binder components are, in particular, thermoplastically processable components.

The preparations preferably contain at least one binder component selected from among:

homo- and copolymers of N-vinyl compounds such as N-vinyllactams, for example N-vinylcaprolactam or N-vinylpiperidone, N-vinylformamide or N-vinylimidazole; in particular homo- and copolymers of N-vinylpyrrolidone (NVP) having K values according to Fikentscher in the region from 10 to 100, preferably 17 to 90, particularly preferably in the region of K 30 (cf. H. Fikentscher, Cellulosechemie 13 (1932), pp. 58-64 and 71-74, such as polyvinylpyrrolidone (PVP), copolymers with vinyl esters, in particular N-vinyl acetate, for example copolymers of 60% by weight of NVP and 40% by weight of vinyl acetate;

acrylate-containing polymers such as polyacrylates, polymethacrylates, copolymers of acrylic acid or of methacrylic acid, in particular their copolymers with alkyl esters of acrylic acid or methacrylic acid such as ethyl acrylate, butyl acrylate or dialkylaminoalkyl esters;

Such polymers are commercially obtainable, for example, under the trade name Eudragit®.

Cellulose derivatives, in particular cellulose esters and cellulose ethers such as alkylcelluloses, for example methylcellulose (\overline{M}ᵣ 20,000 to 150,000) or ethylcellulose, hydroxyalkylcelluloses, for example hydroxypropylcellulose (\overline{M}ᵣ 60,000 to 1.2 million), hydroxyalkylalkylcelluloses, for example hydroxypropylmethylcellulose (\overline{M}ᵣ 10,000 to 150,000), cellulose phthalates, for example cellulose acetate phthalate (\overline{M}ᵣ 40,000);

polyethylene glycols having molecular weights in the range from 400 to 100,000, preferably 4000 to 20,000;

modified starches or starch degradation products such as, for example, maltodextrin;

low-molecular weight matrix components such as sugar alcohols, for example maltitol, mannitol, sorbitol, xylitol, erythritol or isomaltol;

natural or predominantly natural binders such as gelatin, xanthan gum, alginates, polylactides, polyamino acids or mannans, for example galactomannan.

Mixtures of the polymers mentioned can also be employed.

Particularly preferred binders are homo- and copolymers of N-vinylpyrrolidone having K values of 17 to 30, in particular a copolymer with vinyl acetate of the composition VP/VAc 60/40.

The preparations according to the invention can contain the active compound in amounts of from 0.5 to 95% by weight, preferably 5 to 60% by weight, particularly preferably 5 to 25% by weight.

The proportion of excipients can accordingly be 5 to 99.5% by weight, the proportion of matrix-forming binders preferably being 5 to 99.5, particularly preferably 40 to 90, % by weight.

Furthermore, the preparations can additionally contain 0 to 94.5% by weight, preferably 5 to 25% by weight, of customary pharmacologically acceptable excipients, for example surface-active substances such as surfactants, pH-influencing additives, plasticizers, fillers, lubricants, stabilizers such as preservatives or antioxidants, aromas, colorants or flavor-masking substances.

Suitable surfactants are, for example, sucrose esters, alkoxylated fatty alcohols, alkoxylated fatty acids or fatty acid glycerol esters, ethoxylated sorbitan fatty acid esters and polyethoxylated hydrogenated castor oil.

Furthermore suitable are polyoxyethylene/polyoxypropylene block copolymers, which are also known as poloxamers, for example Poloxamer 407 or Poloxamer 338.

As surfactants, the preparations according to the invention preferably contain ethoxylated hydrogenated castor oil, in particular PEG-35 or PEG-40, or poloxamers, in particular Poloxamer 407. These surfactants are preferably employed in amounts of 5 to 15% by weight.

Suitable pH-influencing additives are, for example, organic carboxylic acids or their physiologically acceptable salts—such as, for example, acetic acid, maleic acid, tartaric acid, citric acid—, sugar acids such as, for example, ascorbic acid, amino acids such as, for example, glutamic acid or arginine acid, furthermore inorganic acids or their salts such as, for example, carbonates, hydrogencarbonates, phosphoric acid, hydrogenphosphates or dihydrogenphosphates.

pH-influencing additives employed are in particular citric acid and sodium acetate. These additives can be employed in amounts of 0.1 to 20% by weight, preferably 2 to 5% by weight, based on the total amount of the preparation.

The preparations according to the invention can be produced by spray embedding, spray drying, coprecipitation and lyophilization. Thus it is possible, for example, to dissolve the active compound together with the matrix components in water or an organic solvent such as, for example, methanol, ethanol, isopropanol, methylene chloride, toluene or preferably tetrahydrofuran and subsequently to spray it by single-component nozzles, multicomponent nozzles or via rotating disks.

The preparations according to the invention are preferably produced by melt processes. For this, a homogeneous melt of the substances employed is first prepared, which is then extruded and subjected to shaping. A premixture of all components can be fused or a melt of the excipients can first be produced and the active compounds can then be metered in.

The melt can be produced in suitable devices known per se such as heatable stirring vessels or kneaders at melt temperatures of 40 to 170° C., preferably up to 140° C. The homogeneous melt is then customarily extruded through a nozzle or a perforated plate. The melt is preferably processed in a screw kneader or screw extruder, preferably in a double-screw extruder. The still thermoplastic extrudates emerging from the nozzle or the perforated plate can be shaped, for example, to give granules by customary shaping techniques such as hot- or cold-shaping. The solidified extrudates can also be processed to give granules by means of suitable grinding processes. The still thermoplastic extrudates can also be shaped directly to give tablets by the calendering process known, for example, from EP-A 240 906.

The process is preferably carried out in the absence of water or organic solvents. However, it may be recommended to employ up to 3% by weight of water as a plasticizing additive. If desired, this water can also be removed before the extrusion of the melt by applying a vacuum.

The invention also relates to solid or semisolid, storage-stable pharmaceutical forms, preferably solid pharmaceutical forms for peroral administration.

The preparations according to the invention can be employed as tablets, film-coated tablets, as a filling for hard or soft gelatin capsules or sachets, as granules or beverage granules.

Moreover, the preparations according to the invention can also be employed in nonperoral pharmaceutical forms, such as, for example, suppositories.

Thus ground extrudate can be mixed with the excipients customary for tableting, such as binders, fillers, disintegrants, flow regulators or mold release agents and then pressed in a conventional tablet press to give tablets. The particle size of the ground extrudate is preferably <1500 μm. The tableting mixture can also contain a matrix release-delaying agent.

The tablets can also be provided with a film coating. In this way, enteric film-coated tablets can also be produced, or film-coated tablets having a coating delaying the release of active compound, for example a coating containing release-delaying polymers of the Eudragit type.

The preparations according to the invention can also be filled into hard gelatin capsules or into sachets as a powder mixture with customary excipients or serve as a filling for soft gelatin capsules.

The preparations according to the invention are in this case employed in amounts such that, per dose unit, typically 2.5 to 200 mg, preferably 2.5 to 20 mg, of torasemide are present.

It is also possible to prepare combination pharmaceutical forms with further diuretics, for example with furosemide, hydrochlorothiazide, amiloride, triamterene and spironolactone.

With the aid of the preparations according to the invention, storage-stable pharmaceutical forms can be prepared in which the torasemide is present in noncrystalline form and preferably as a solid solution or in amorphous form. With the aid of DSC measurements (Differential Scanning Calorimetry) or WAXS recordings (wide angle X-ray spectroscopy, wide-angle X-ray scattering), it can be shown that the preparations have no crystalline fractions. In this way, the problem of the differing stability and bioavailability of the polymorphic forms can be avoided.

### EXAMPLES

### Examples 1 to 6

The mixtures listed in Table I were fused and extruded in a single-screw extruder (screw length 170 mm, screw diameter 6.4 mm). The three heating zones of the extruder had the following temperature profile: zone 1: 65 to 76° C., zone 2: 100 to 130° C., zone 3: 110 to 140° C. The speed of rotation of the screw was 120 to 180 rpm.

The homogeneous extrudates emerging from the nozzle were ground in the solidified state to give granules having a particle size of <1500 μm.

TABLE I
(data in % by weight)

Formulation No.

| | 1 | 2 | 3 | 4 | 5 | 6 |
|---|---|---|---|---|---|---|
| Torasemide | 20 | 20 | 20 | 20 | 20 | 20 |
| Kollidon VA 64 | 75 | 75 | 75 | 70 | 70 | |
| Kollidon K17 | | | | | | 75 |
| Citric acid anh. | | | | 5 | | |
| Cremophor ® RH 40 | 5 | | | | | |
| Sodium acetate anh. | | | | | 5 | |
| Poloxamer 407 | | 5 | | 5 | 5 | 5 |
| Sucrose monopalmitate | | | 5 | | | |

Kollidon ® VA 64: Copolymer of 60% by weight N-vinylpyrrolidone and 40% by weight of vinyl acetate, BASF
Kollidon K17: Polyvinylpyrrolidone, K value 17, BASF
Cremophor RH 40: PEG-40-hydrogenated castor oil, BASF

### Example 7

Production of Tablets

Ground extrudate (as described in formulations Nos. 1 to 6) is mixed in the appropriate amounts with the excipients indicated below and pressed to give curved tablets having a weight of 200 mg and a diameter of 8 mm.

| | |
|---|---|
| Formulation 1 | 50 mg |
| Crosscarmelose | 10 mg |
| CaHPO₄ anhydr. | 136 mg |
| Aerosil 200⁺⁾ | 2 mg |
| Magnesium stearate | 2 mg |

⁺⁾highly pure silica; BET surface area 200 ± 25 m², mean size of the primary particles 12 nm

### Example 8

Production of Matrix Delayed-Release Tablets

Analogously to Example 7, tablets of the following composition are produced:

| | |
|---|---|
| Formulation 1 | 50 mg |
| CaHPO₄ | 133 mg |
| Crosscarmelose | 5 mg |
| Eudragit RL⁺⁾ | 8 mg |
| Aerosil 200 | 2 mg |
| Magnesium stearate | 2 mg |

⁺⁾Eudragit RL: poly(ethyl acrylate, methyl methacrylate, trimethylammonium methacrylate chloride (1:2:0.2), Roehm

### Example 9

Production of Film-Coated Tablets Having Modified Release of Active Compound

The tablets according to Example 7 are provided with a film coating in a coater by means of a conventional spraying process.

| | |
|---|---|
| Tablet weight | 204.55 mg |
| Composition of the coating: | |
| Eudragit L100-55 | 2.28 mg |
| Polyoxyethylene-20-sorbitan monooleate | 0.04 mg |
| NaOH | 0.03 mg |
| Simethicone | 0.01 mg |
| Talc | 1.52 mg |
| Macrogol 6000 | 0.67 mg |

### Example 10

Hard Gelatin Capsules; Sachets

Hard gelatin capsules of size 1 or sachets are filled with 100 mg of the homogeneous powder mixture of the following composition:

| | |
|---|---|
| Formulation 1 | 40 mg |
| Mannitol | 49.5 mg |
| Aerosil 200 | 0.5 mg |

The WAXS recordings of torasemide as a crystalline crude material (upper line) and of torasemide extrudate (lower line) are illustrated in the figure.

We claim:

1. A stable solid or semisolid pharmaceutical preparation, comprising torasemide in essentially noncrystalline form.

2. A preparation as claimed in claim 1, comprising at least one binder component and, if appropriate, further pharmaceutically acceptable excipients.

3. A preparation as claimed in claim 1, comprising torasemide in the form of a solid solution in a binder matrix.

4. A preparation as claimed in claim 1 or 2, in which the torasemide is present in the form of amorphous agglomerates.

5. A preparation as claimed in one of claims 1 to 4, comprising at least one binder component selected from the group consisting of homo- and copolymers of N-vinylpyrrolidone.

6. A preparation as claimed in one of claims 1 to 5, comprising
a) 0.5 to 95% by weight of torasemide
b) 5 to 99.5% by weight of at least one binder component
c) 0 to 94.5% by weight of further pharmaceutically acceptable excipients, the sum of the components a), b) and, if appropriate, c) being 100% by weight.

7. A preparation as claimed in one of claims 1 to 6, comprising 0.1 to 20% by weight of a surfactant.

8. A preparation as claimed in one of claims 1 to 7, comprising 0.1 to 20% by weight of a pH-stabilizing compound.

9. A process for the production of a pharmaceutical preparation as claimed in one of claims 1 to 8, which comprises homogeneously mixing torasemide with at least one binder component and, if appropriate, further pharmaceutically acceptable excipients in the melt and subsequently extruding.

10. A storage-stable pharmaceutical form for administration, comprising preparations as claimed in one of claims 1 to 8.
